(19) United States
(12) Patent Application Publication
(10) Pub. No.: US 2008/0267861 A1
(43) Pub. Date: Oct. 30, 2008

(54) Myocardial Perfusion Imaging

(75) Inventors: Hsiao D. Lieu, Burlingame, CA (US); Gregory Thomas, Dana Point, CA (US)

Correspondence Address: MCDONNELL BOEHNEN HULBERT & BERGHOFF LLP, 300 S. WACKER DRIVE, 32ND FLOOR, CHICAGO, IL, 60606, US

(73) Assignee: CV THERAPEUTICS, INC., Palo Alto, CA (US)

(21) Appl. No.: 11/969,047

(22) Filed: Jan. 3, 2008

Related U.S. Application Data
(60) Provisional application No. 60/878,529, filed on Jan. 3, 2007.

(51) Int. Cl. A61K 51/00; A61K 31/7076

(52) U.S. Cl. 424/1.11; 514/46

(57) ABSTRACT

This invention relates to methods for performing myocardial perfusion imaging for diagnosing and characterizing coronary artery disease using an intravenous (IV) bolus injection of regadenoson while the patient is undergoing sub-maximal exercise.

### RELATED APPLICATION

This application claims priority to U.S. Provisional Patent Application Ser. No. 60/878,529, filed Jan. 3, 2007, the entirety of which is incorporated herein by reference.

### FIELD OF THE INVENTION

This invention relates to methods for performing myocardial perfusion imaging for diagnosing and characterizing coronary artery disease using an intravenous (IV) bolus injection of regadenoson while the patient is undergoing low-level exercise.

### BACKGROUND

Myocardial perfusion imaging (MPI) with radionuclide agents is an integral part of cardiology practice for diagnosing and characterizing coronary artery disease [See, Verani et al. (1994) Am J Cardiac Imaging 8: 223-230; Ritchie et al. (1995) J Am Coll Cardiol 25: 521-527; Gibbons et al. (1999). J Am Coll Cardiol 33: 2092-2197; Braunwald et al. (2000) J Am Coll Cardiol 36: 970-1062; and Eagle et al. (1996). J Am Coll Cardiol 27: 910-948].

MPI is a non-invasive technique based on the principle that radiopharmaceuticals, such as ²⁰¹Thallium, ^{99m}Technetium-sestaribi and ^{99m}Technetium-tetrofosmin distribute according to blood flow. The imaging protocol requires that two sets of images are obtained: one obtained at rest and a second obtained under conditions that increase coronary blood flow (“stress scan”), such as exercise or the administration of a pharmacological stress agent (e.g., a coronary vasodilator). Pharmacological stress agents are used in patients who are unable to exercise sufficiently. These agents increase coronary blood flow by vasodilating the coronary arteries.

In 2005, almost 4.3 million or 46% of patients who underwent stress MPI in the U.S. were tested with the pharmacological agents adenosine and dipyridamole (both vasodilators), or the inotropic agent dobutamine (Nuclear Medicine Market Summary Report. November 2006. IMv Medical Information Division, Inc.) The most frequent reasons for using pharmacological stress in place of exercise are orthopedic problems, chronotropic incompetence, deconditioning, left bundle branch block or right ventricular pacing and occasionally, secondary to the inability to stop relevant medications.

Adenosine, dipyridarnole and dobutamine are administered as short infusions, followed by administration of a radiopharmaceutical. These agents are less than ideal as they are associated with undesirable side effects (Belardinelli et al. 1998. J Pharmacol Exp Ther 284:1066-1073; Shryock et al. 1998 Circulation 98:711-718)

Adenosine induces coronary vasodilatation and enhancement of coronary blood flow by activating coronary A_{2A} adenosine receptors. Adenosine has a half-life of less than 10 seconds in vivo and therefore blood flow returns rapidly to the resting state after cessation of adenosine administration. For these reasons, adenosine is administered as a continuous infusion. In addition to its activity via the A_{2A} receptor, adenosine is known to activate three other adenosine receptor subtypes (A₁, A_{2B} and A₃) which contribute to the side effect profile (including the potential to cause atrioventricular block and bronchospasm) [Adenoscan (adenosine) Package Insert (September, 2000). Adverse Reactions. Fujisawa Healthcare, Inc., Deerfield Ill.; Feoktistov et al. 1997. Am Soc Pharmacol and Exp Ther 49:381-402]

Dipyridamole, a nucleoside transport inhibitor, increases plasma and tissue levels of adenosine by inhibition of its transport into the cells, thereby reducing its clearance. The side effects of dipyridamole may persist for long periods of time (hours) because dipyridamnole has a half-life that is longer than that of adenosine. Because of the longer duration of action of dipyridamole, optimal monitoring of the patients for delayed side effects requires ongoing observation after the procedure.

Multiple studies have found that combining exercise with adenosine testing (“AdenoEx”) improves image quality, decreases adverse effects and improves patient acceptance (Thomas et al., 2000, J Nucl Cardiol; 7(5):439-46). In addition, there is evidence that sensitivity for the detection of coronary artery disease is also improved [Thomas et al., 2004, Am J Cardiol. 94(2A):3D-10D. Discussion 10D-11D; Samady et al. 2002, J Nucl Cardiol, 9:188-196; Hashimoto et al. 1999, J Nucl Cardio, 6:612-619; and Pennell et al. 1995, J Am Coll Cardio, 25:1300-1309]. In most luminary laboratories, AdenoEx has become the standard of care (Thomas et al., 2004, Am J. Cardiol. 94(2A):3D-10D. Discussion 10D-11D).

Although vasodilators are combined with exercise in approximately 17% of MPI studies in the United States (Division IMI. Nuclear Medicine Census Market Summary Reports. Greenbelt, Md., 2006) and, indeed, combination testing is recommended by the American Society of Nuclear Cardiology practice guidelines (Henzlova et al. 2006, “Stress protocols and tracers”. In: DePeuy E G, ed. Imaging Guidelines for Nuclear Cardiology Procedures: A Report from the Nuclear Cardiology Quality Assurance Committee: American Society of Nuclear Cardiology:171), the Food and Drug Administration (FDA) labeled indications for adenosine and dipyridamole do not include use with exercise.

New and potent partial A_{2A} agonists that increase CBF but do not significantly increase peripheral blood flow have been identified. The partial A_{2A} agonists, and especially Regadenoson and CVT-3033 have a rapid onset and a short duration when administered. An unexpected and newly identified benefit of these new compounds is that they are very useful when administered in a very small quantity in a single bolus intravenous injection. The partial A_{2A} receptor agonists can be administered in amounts as little as 10 μg and as high as 600 μg or more and still be effective few if any side-effects. An optimal intravenous dose will include from about 100 to about 500 μg of at least one partial A_{2A} receptor agonist. This amount is unexpectedly small when compared with adenosine which is typically administered in continuously by IV at a rate of about 140 μg/kg/min. Unlike adenosine, the same dosage of partial A_{2A} receptor agonists, an in particular, Regadenoson and CVT-3033 can be administered to a human patient regardless of the patient's weight. Thus, the administration of a single uniform amount of a partial A_{2A} receptor agonists by iv bolus for myocardial imaging is dramatically simpler and less error prone than the time and weight dependent administration of adenosine.

It has now been discovered that partial A_{2A} agonists not only are suitable and safe for use in conjunction with exercise, given the fact that they are administered by single bolus dosing independent of patient weight, they provide unique benefits in this type of diagnostic treatment.

### SUMMARY OF THE INVENTION

The following are aspects of this invention:

A method of diagnosing myocardial dysfunction during vasodilator induced myocardial stress perfusion imaging in a human patient, comprising administering at least 10 μg of at least one partial A_{2A} adenosine receptor agonist to the mammal while the patient is undergoing sub-maximal exercise.

A method of diagnosing myocardial dysfunction during vasodilator induced myocardial stress perfusion imaging in a human patient, comprising administering no more than about 1000 μg of a partial A_{2A} adenosine receptor agonist to the patient while the patient is undergoing sub-maximal exercise.

A method of diagnosing myocardial dysfunction during vasodilator induced myocardial stress perfusion imaging in a human patient, comprising administering a partial A_{2A} adenosine receptor agonist in an amount ranging from about 10 to about 600 μg to the patient while the patient is undergoing sub-maximal exercise.

A method of diagnosing myocardial dysfunction during vasodilator induced myocardial stress perfusion imaging in a human patient, comprising administering a radionuclide and a partial A_{2A} adenosine receptor agonist in an amount ranging from about 10 to about 600 μg wherein the A_{2A} adenosine receptor is administered in a single dose while the patient is undergoing sub-maximal exercise.

A method of diagnosing myocardial dysfunction during vasodilator induced myocardial stress perfusion imaging in a human patient, comprising administering a radionuclide and a partial A_{2A} adenosine receptor agonist in an amount ranging from about 10 to about 600 μg wherein the partial A_{2A} adenosine receptor agonist is administered by iv bolus while the patient is undergoing sub-maximal exercise.

A method of diagnosing myocardial dysfunction during vasodilator induced myocardial stress perfusion imaging in a human patient, comprising administering a radionuclide and a partial A_{2A} adenosine receptor agonist in an amount ranging from about 10 to about 600 μg wherein the partial wherein the partial A_{2A} adenosine receptor agonist is administered in less than about 10 seconds while the patient is undergoing sub-maximal exercise.

A method of diagnosing myocardial dysfunction during vasodilator induced myocardial stress perfusion imaging in a human patient, comprising administering a radionuclide and a partial A_{2A} adenosine receptor agonist in an amount ranging from about 10 to about 600 μg wherein the partial A_{2A} adenosine receptor agonist is administered in an amount greater than about 10 μg while the patient is undergoing sub-maximal exercise.

A method of diagnosing myocardial dysfunction during vasodilator induced myocardial stress perfusion imaging in a human patient, comprising administering a radionuclide and a partial A_{2A} adenosine receptor agonist in an amount ranging from about 10 to about 600 μg wherein the partial A_{2A} adenosine receptor agonist is administered in an amount greater than about 100 μg while the patient is undergoing sub-maximal exercise.

A method of diagnosing myocardial dysfunction during vasodilator induced myocardial stress perfusion imaging in a human patient, comprising administering a radionuclide and a partial A_{2A} adenosine receptor agonist in an amount ranging from about 10 to about 600 μg wherein the partial A_{2A} adenosine receptor agonist is administered in an amount no greater than 600 μg while the patient is undergoing sub-maximal exercise.

A method of diagnosing myocardial dysfunction during vasodilator induced myocardial stress perfusion imaging in a human patient, comprising administering a radionuclide and a partial A_{2A} adenosine receptor agonist in an amount ranging from about 10 to about 600 μg wherein the partial A_{2A} adenosine receptor agonist is administered in an amount no greater than 500 μg while the patient is undergoing sub-maximal exercise.

A method of diagnosing myocardial dysfunction during vasodilator induced myocardial stress perfusion imaging in a human patient, comprising administering a radionuclide and a partial A_{2A} adenosine receptor agonist in an amount ranging from about 10 to about 600 μg while the patient is undergoing sub-maximal exercise, wherein the partial A_{2A} adenosine receptor agonist is administered in an amount ranging from about 100 μg to about 500 μg.

A method of diagnosing myocardial dysfunction during vasodilator induced myocardial stress perfusion imaging in a human patient, comprising administering a radionuclide and a partial A_{2A} adenosine receptor agonist in an amount ranging from about 10 to about 600 μg while the patient is undergoing sub-maximal exercise, wherein the partial A_{2A} adenosine receptor agonist is selected from the group consisting of CVT-3033, Regadenoson, and combinations thereof.

A method of diagnosing myocardial dysfunction during vasodilator induced myocardial stress perfusion imaging in a human patient, comprising administering a radionuclide and a partial A_{2A} adenosine receptor agonist in an amount ranging from about 10 to about 600 μg while the patient is undergoing sub-maximal exercise, wherein the myocardium is examined for areas of insufficient blood flow following administration of the radionuclide and the partial A_{2A} adenosine receptor agonist.

A method of diagnosing myocardial dysfunction during vasodilator induced myocardial stress perfusion imaging in a human patient, comprising administering a radionuclide and a partial A_{2A} adenosine receptor agonist in an amount ranging from about 10 to about 600 μg while the patient is undergoing sub-maximal exercise, wherein the myocardium is examined for areas of insufficient blood flow following administration of the radionuclide and the partial A_{2A} adenosine receptor agonist wherein the myocardium examination begins within about 1 minute from the time the partial A_{2A} adenosine receptor agonist is administered.

A method of diagnosing myocardial dysfunction during vasodilator induced myocardial stress perfusion imaging in a human patient, comprising administering a radionuclide and a partial A_{2A} adenosine receptor agonist in an amount ranging from about 10 to about 600 μg while the patient is undergoing sub-maximal exercise, wherein the administration of the partial A_{2A} adenosine receptor agonist causes at least a 2.5 fold increase in coronary blood flow.

A method of diagnosing myocardial dysfunction during vasodilator induced myocardial stress perfusion imaging in a human patient, comprising administering a radionuclide and a partial A_{2A} adenosine receptor agonist in an amount ranging from about 10 to about 600 μg while the patient is undergoing sub-maximal exercise, wherein the administration of the partial A_{2A} adenosine receptor agonist causes at least a 2.5 fold increase in coronary blood flow that is achieved within about 1 minute from the administration of the partial A_{2A} adenosine receptor agonist.

A method of diagnosing myocardial dysfunction during vasodilator induced myocardial stress perfusion imaging in a human patient, comprising administering a radionuclide and a partial A_{2A} adenosine receptor agonist in an amount ranging from about 10 to about 600 μg while the patient is undergoing sub-maximal exercise, wherein the radionuclide and the partial A_{2A} adenosine receptor agonist are administered separately.

A method of diagnosing myocardial dysfunction during vasodilator induced myocardial stress perfusion imaging in a human patient, comprising administering a radionuclide and a partial A_{2A} adenosine receptor agonist in an amount ranging from about 10 to about 600 μg while the patient is undergoing sub-maximal exercise, wherein the radionuclide and the partial A_{2A} adenosine receptor agonist are administered simultaneously.

A method of diagnosing myocardial dysfunction during vasodilator induced myocardial stress perfusion imaging in a human patient, comprising administering a radionuclide and a partial A_{2A} adenosine receptor agonist in an amount ranging from about 10 to about 600 μg while the patient is undergoing sub-maximal exercise, wherein the administration of the partial A_{2A} adenosine receptor agonist causes at least a 2.5 fold increase in coronary blood flow for less than about 5 minutes.

A method of diagnosing myocardial dysfunction during vasodilator induced myocardial stress perfusion imaging in a human patient, comprising administering a radionuclide and a partial A_{2A} adenosine receptor agonist in an amount ranging from about 10 to about 600 μg while the patient is undergoing sub-maximal exercise, wherein the administration of the partial A_{2A} adenosine receptor agonist causes at least a 2.5 fold increase in coronary blood flow for less than about 3 minutes.

A method of diagnosing myocardial dysfunction during vasodilator induced myocardial stress perfusion imaging in a human patient, comprising administering Regadenoson in an amount ranging from about 10 to about 600 μg in a single iv bolus while the patient is undergoing sub-maximal exercise.

A method of diagnosing myocardial dysfunction during vasodilator induced myocardial stress perfusion imaging in a human patient, comprising administering Regadenoson in an amount ranging from about 100 to about 500 μg in a single iv bolus while the patient is undergoing sub-maximal exercise.

In all of the methods above, the dose is typically administered in a single iv bolus.

In all of the methods above, at least one radionuclide is administered before, with or after the administration of the A_{2A} adenosine receptor agonist to facilitate myocardial imaging.

In all of the methods, the myocardial dysfunction includes coronary artery disease, coronary artery dilation, ventricular dysfanction, differences in blood flow through disease free coronary vessels and stenotic vessels, or a combination thereof.

In all of the methods, the method of myocardial stress perfusion imaging is a noninvasive imaging procedure. The imaging can be performed by methods including scintigraphy, single photon emission computed tomography (SPECT), positron emission tomography (PET), nuclear magnetic resonance (NMR) imaging, perfusion contrast echocardiography, digital subtraction angiography (DSA), and ultra fast X-ray computed tomography (CINE CT), and combinations of these techniques.

In certain embodiments of the method of myocardial stress perfusion imaging, the step of detecting myocardial dysfunction comprises measuring coronary blood flow velocity on the human patient to assess the vasodilatory capacity of diseased coronary vessels as compared with disease free coronary vessels.

In other embodiments of the method of myocardial stress perfusion imaging, the step of detecting myocardial dysfunction comprises assessing the vasodilatory capacity (reserve capacity) of diseased coronary vessels as compared with disease-free coronary vessels.

### DESCRIPTION OF THE FIGURES

FIG. 1 illustrates heart-to-background ratios following AdenoSup and RegEx. Data are from the 39 patients who crossed over after receiving adenosine while supine (AdenoSup) to regadenoson during low-level exercise (RegEx). Data presented are means ±SD. P-values are for differences between AdenoSup and RegEx (Wilcoxon matched pairs Signed Rank test)

FIG. 2 displays a side-by-side comparison of the overall image quality between AdenoSup and RegEx scans. Data are from the 39 patients who underwent adenosine while supine (AdenoSup) and regadenoson during low-level exercise (RegEx). P-values are for differences between AdenoSup and RegEx (Sign Test, ignoring the “same” category).

FIG. 3 presents a side-by-side comparison of the image quality with respect to subdiagphragmatic interference between AdenoSup and RegEx scans. Data are from the 39 patients who received adenosine while supine (AdenoSup) and regadenoson during low-level exercise (RegEx). P-values are for differences between AdenoSup and RegEx (Sign Test, ignoring the “same” category).

FIG. 4 is a representative example of the difference in image quality and heart-to-gut ratio in the same patient undergoing adenosine supine myocardial perfusion imaging (AdenoSup) and low-level exercise with regadenoson (RegEx).

FIG. 5 shows the results of a questionnaire on patient preference for RegEx and PlcEx in comparison to AdenoSup. Following the exercise test, all 60 patients were asked “How did the exercise test compare to the test when you were lying down?” The p-value is a comparison of the responses in the RegEx group and PIcEx group (Cochran-Mantel-Haenszel).

FIG. 6A shows the effect of AdenoSup, RegEx, and PlcEx on heart rate. Data points shown represent means ±SEM. At 4, 6, 8, 10, 14, and 24 minutes following the start of exercise (time 0), p-values comparing mean heart rate during regadenoson administration during exercise (RegEx) vs. placebo (PlcEx) administration during exercise were <0.05. (AdenoSup time points were slightly different than those for RegEx and PlcEx; therefore, comparisons at individual time points were not possible).

FIG. 6B shows the effect of AdenoSup, RegEx, and PlcEx on systolic blood pressure. Data points shown represent means ±SEM. P-values for all comparisons between RegEx and PlcEx were >0.05 at all time points. (AdenoSup time points were slightly different than those for RegEx and PlcEx; therefore, comparisons at individual time points were not possible).

### DETAILED DESCRIPTION OF THE INVENTION

Sub-maximal exercise during pharmacologic myocardial perfusion imaging (MPI) decreases adverse effects and improves patient acceptance, image quality, and may increase the sensitivity for detecting perfusion defects. Regadenoson and other partial adenosine A_{2A} receptor agonists are under active investigation as pharmacologic stress MPI agents and have now been found to be safe and efficacious when combined with sub-maximal exercise on pharmacologic MPI.

In some embodiments of the invention, myocardial dysfunction is detected by myocardial perfusion imaging. The imaging can be performed by methods including scintigraphy, single photon emission computed tomography (SPECT), positron emission tomography (PET), nuclear magnetic resonance (NMR) imaging, perfusion contrast echocardiography, digital subtraction angiography (DSA), and ultra fast X-ray computed tomography (CINE CT), and combinations of these techniques.

The partial A_{2A} adenosine receptor agonists can be administered in amounts as little as 10 μg and as high as 600 μg or more and still be effective with few if any side-effects. An optimal intravenous dose will include from about 100 to about 500 μg of at least one partial A_{2A} adenosine receptor agonist. This amount is unexpectedly small when compared with adenosine which is typically administered in continuously by iv infusion at a rate of about 140 μg/kg/min. Unlike adenosine, the same dosage of partial A_{2A} adenosine receptor agonists, an in particular, Regadenoson and CVT-3033 can be administered to a human patient regardless of the patient's weight. Thus, the administration of a single uniform amount of a partial A_{2A} adenosine receptor agonist by iv bolus for myocardial imaging is dramatically simpler and less error prone than the time and weight dependent administration of adenosine.

Pharmaceutical compositions including the compounds of this invention, and/or derivatives thereof, may be formulated as solutions or lyophilized powders for parenteral administration. Powders may be reconstituted by addition of a suitable diluent or other pharmaceutically acceptable carrier prior to use. If used in liquid form the compositions of this invention are preferably incorporated into a buffered, isotonic, aqueous solution. Examples of suitable diluents are normal isotonic saline solution, standard 5% dextrose in water and buffered sodium or ammonium acetate solution. Such liquid formulations are suitable for parenteral administration, but may also be used for oral administration. It may be desirable to add excipients such as polyvinylpyrrolidinone, gelatin, hydroxy cellulose, acacia, polyethylene glycol, mannitol, sodium chloride, sodium citrate or any other excipient known to one of slill in the art to pharmaceutical compositions including compounds of this invention. Further compositions can be found in U.S published application 2005/0020915, the specification of which is incorporated herein by reference in its entirety.

A first class of compounds that are potent and selective agonists for the A2A adenosine receptor that are useful in the methods of this invention are 2-adenosine N-pyrazole compounds having the formula:

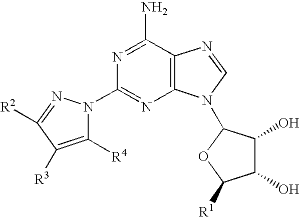

wherein

R¹═CH₂OH, —CONR⁵R⁶;

R² and R⁴ are selected from the group consisting of H, C₁₋₆ alkyl and aryl, wherein the alkyl and aryl substituents are optionally substituted with halo, CN, CF₃, OR²⁰ and N(R²⁰)₂ with the proviso that when R² is not hydrogen then R⁴ is hydrogen, and when R⁴ is not hydrogen then R² is hydrogen;

R³ is independently selected from the group consisting of C₁₋₁₅ alkyl, halo, NO₂, CF₃, CN, OR²⁰, SR²⁰, N(R²⁰)₂, S(O)R²², SO₂R²², SO₂N(R²⁰)₂, SO₂NR²⁰COR²², SO₂NR²⁰CO₂R²², SO₂NR²⁰CON(R²⁰)₂, N(R²⁰)₂ NR²⁰COR²², NR²⁰CO₂R²², NR²⁰CON(R²⁰)₂, NR²⁰C(NR²⁰)NHR²³, COR²⁰, CO₂R²⁰, CON(R²⁰)₂, CONR²⁰SO₂R²², NR²⁰SO₂R²², SO₂NR²⁰CO₂R²², OCONR²⁰SO₂R²², OC(O)R²⁰, C(O)OCH₂OC(O)R²⁰, and OCON(R²⁰)₂, —CONR⁷R⁸, C₂₋₁₅ alkenyl, C₂₋₁₅ alkynyl, heterocyclyl, aryl, and heteroaryl, wherein the alkyl, alkenyl, alkynyl, aryl, heterocyclyl and heteroaryl substituents are optionally substituted with from 1 to 3 substituents independently selected from the group consisting of halo, alkyl, NO₂, heterocyclyl, aryl, heteroaryl, CF₃, CN, OR²⁰, SR²⁰, N(R²⁰)₂, S(O)R²², SO₂R²², SO₂N(R²⁰)₂, SO₂NR²⁰COR²², SO₂NR²⁰CO₂R²², SO₂NR²⁰CON(R²⁰)₂, N(R²⁰)₂ NR²⁰COR²², NR²⁰CO₂R²², NR²⁰CON(R²⁰)₂, NR²⁰C(NR²⁰)NHR²³, COR²⁰, CO₂R²⁰, CON(R²⁰)₂, CONR²⁰SO₂R², NR²⁰SO₂R²², SO₂NR²⁰CO₂R²², OCONR²⁰SO₂R²², OC(O)R²⁰, C(O)OCH₂OC(O)R²⁰, and OCON(R²⁰)₂ and wherein the optional substituted heteroaryl, aryl, and heterocyclyl substituents are optionally substituted with halo, NO₂, alkyl, CF₃, amino, mono- or di-alkylamino, alkyl or aryl or heteroaryl amide, NCOR²², NR²⁰SO₂R²², COR²⁰, CO₂R²⁰, CON(R²⁰)₂, NR²⁰CON(R²⁰)₂, OC(O)R²⁰, OC(O)N(R²⁰)₂, SR²⁰, S(O)R²², SO₂R²², SO₂N(R²⁰)₂, CN, or OR²⁰;

R⁵ and R⁶ are each individually selected from H, and C₁-C₁₅ alkyl that is optionally substituted with from 1 to 2 substituents independently selected from the group of halo, NO₂, heterocyclyl, aryl, heteroaryl, CF₃, CN, OR²⁰, SR²⁰, N(R²⁰)₂, S(O)R²², SO₂R²², SO₂N(R²⁰)₂, SO₂NR²⁰COR²², SO₂NR²⁰CO₂R²², SO₂NR²⁰CON(R²⁰)₂, N(R²⁰)₂NR²⁰COR²², NR²⁰CO₂R²², NR²⁰CON(R²⁰)₂, NR²⁰C(NR²⁰)NHR²³, COR²⁰, CO₂R²⁰, CON(R²⁰)₂, CONR²⁰SO₂R²², NR²⁰SO₂R²², SO₂NR²⁰CO₂R²², OCONR²⁰SO₂R²², OC(O)R²⁰, C(O)OCH₂OC(O)R²⁰, and OCON(R²⁰)₂ wherein each optional substituted heteroaryl, aryl, and heterocyclyl substituent is optionally substituted with halo, NO₂, alkyl, CF₃, amino, monoalkylamino, dialkylamino, alkylamide, arylamide, heteroarylamide, NCOR²², NR²⁰SO₂R²², COR²⁰, CO₂R²⁰, CON(R²⁰)₂, NR²⁰CON(R²⁰)₂, OC(O)R²⁰, OC(O)N(R²⁰)₂, SR²⁰, S(O)R²², SO₂R²², SO₂N(R²⁰)₂, CN, and OR²⁰;

R⁷ and R³ are each independently selected from the group consisting of hydrogen, C₁₋₁₅ alkyl, C₂₋₁₅ alkenyl, C₂₋₁₅ alkynyl, heterocyclyl, aryl and heteroaryl, wherein the alkyl, alkenyl, alkynyl, aryl, heterocyclyl and heteroaryl substituents are optionally substituted with from 1 to 3 substituents independently selected from the group of halo, NO₂, heterocyclyl, aryl, heteroaryl, CF₃, CN, OR²⁰, SR²⁰, N(R²⁰)₂, S(O)R²², SO₂R²², SO₂N(R²²)₂, SO₂NR²⁰CO₂R²², SO₂NR²⁰CO₂R²², SO₂NR²⁰CON(R²⁰)₂, N(R²⁰)₂NR²⁰COR²², NR²⁰CO₂ R²², NR²⁰CON(R²⁰)₂, NR²⁰C(NR²⁰)NHR²³, COR²⁰, CO₂R²⁰, CON(R²⁰)₂, CON²⁰SO₂R²², NR²⁰SO₂R²², SO₂NR²⁰CO₂R², OCONR²⁰SO₂R²², OC(O)R²⁰, C(O)OCH₂OC(O)R²⁰ and OCON(R²⁰)₂ and wherein each optional substituted heteroaryl, aryl and heterocyclyl substituent is optionally substituted with halo, NO₂, alkyl, CF₃, amino, mono- or di-alkylamino, alkyl or aryl or heteroaryl amide, NCOR¹², NR²⁰SO₂R²², COR²⁰, CO₂R²⁰, CON(R)₂, NR²⁰CON(R²⁰)₂, OC(O)R²⁰, OC(O)N(R²⁰)₂, SR²⁰, S(O)R²², SO₂R²², SO₂N(R²⁰)₂, CN, and OR²⁰;

R²⁰ is selected from the group consisting of H, C₁₋₁₅ alkyl, C₂₋₁₅ alkenyl, C₂₋₁₅ alkynyl, heterocyclyl, aryl, and heteroaryl, wherein the alkyl, alkenyl, alkynyl, heterocyclyl, aryl, and heteroaryl substituents are optionally substituted with from 1 to 3 substituents independently selected from halo, alkyl, mono- or dialkylamino, alkyl or aryl or heteroaryl amide, CN, O—C₁₋₆ alkyl, CF₃, aryl, and heteroaryl; and

R²² is selected from the group consisting of C₁₋₁₅ alkyl, C₂₋₁₅ alkenyl, C₂₋₁₅ alkynyl, heterocyclyl, aryl, and heteroaryl, wherein the alkyl, alkenyl, alkynyl, heterocyclyl, aryl, and heteroaryl substituents are optionally substituted with from 1 to 3 substituents independently selected from halo, alkyl, mono- or dialkylamino, alkyl or aryl or heteroaryl amide, CN, O—C₁₋₆ alkyl, CF₃, aryl, and heteroaryl.

In an related group of compounds of this invention,
R³ is selected from the group consisting of C₁₋₁₅ alkyl, halo, CF₃, CN, OR¹⁰, SR²⁰, S(O)R²², SO₂R²², SO₂N(R²⁰)₂, COR²⁰, CO₂R²⁰, —CONR⁷R⁸, aryl and heteroaryl wherein the alkyl, aryl and heteroaryl substituents are optionally substituted with from 1 to 3 substituents independently selected from the group consisting of halo, aryl, heteroaryl, CF₃, CN, OR²⁰, SR²⁰, S(O)R²², SO₂R²², SO₂N(R²⁰)₂, COR²⁰, CO₂R²⁰ or CON(R²⁰)₂, and each optional heteroaryl and aryl substituent is optionally substituted with halo, alkyl, CF₃ CN, and OR²⁰;
R⁵ and R⁶ are independently selected from the group of H and C₁-C₁₅ alkyl including one optional aryl substituent and each optional aryl substituent that is optionally substituted with halo or CF₃;
R⁷ is selected from the group consisting of C₁₋₁₅ alkyl, C₂₋₁₅ alkynyl, aryl, and heteroaryl, wherein the alkyl, alkynyl, aryl, and heteroaryl substituents are optionally substituted with from 1 to 3 substituents independently selected from the group consisting of halo, aryl, heteroaryl, CF₃, CN, OR²⁰, and each optional heteroaryl and aryl substituent is optionally substituted with halo, alkyl, CF₃ CN, or OR²⁰;
R⁸ is selected from the group consisting of hydrogen and C₁₋₁₅ alkyl;
R²⁰ is selected from the group consisting of H, C₁₋₄ alkyl and aryl, wherein alkyl and aryl substituents are optionally substituted with one alkyl substituent; and
R²² is selected from the group consisting of C₁₋₄ alkyl and aryl which are each optionally substituted with from 1 to 3 alkyl group.

In yet another related class of compounds,
R¹ is CH₂OH;
R³ is selected from the group consisting of CO₂R²⁰, —CONR⁷R⁸ and aryl where the aryl substituent is optionally substituted with from 1 to 2 substituents independently selected from the group consisting of halo, C₁₋₆ alkyl, CF₃ and OR²⁰;
R⁷ is selected from the group consisting of hydrogen, C₁₋₈ alkyl and aryl, where the alkyl and aryl substituents are optionally substituted with one substituent selected from the group consisting of halo, aryl, CF₃, CN, OR²⁰ and wherein each optional aryl substituent is optionally substituted with halo, alkyl, CF₃ CN, and OR²⁰;
R⁸ is selected from the group consisting of hydrogen and C₁₋₈ alkyl; and
R²⁰ is selected from hydrogen and Cl₄ alkyl.

In a still another related class of compounds of this invention,
R¹═CH₂OH;
R³ is selected from the group consisting of CO₂R²⁰, —CONR⁷R⁸, and aryl that is optionally substituted with one substituent selected from the group consisting of halo, C₁₋₃ alkyl and OR²⁰;
R⁷ is selected from of hydrogen, and C₁₋₃ alkyl;
R⁸ is hydrogen; and
R²⁰ is selected from hydrogen and C₁₋₄ alkyl.
   In this preferred embodiment, R³ is most preferably selected from —CO₂Et and —CONHEt.

In yet another related class of compounds,
R¹═—CONHEt,
R³ is selected from the group consisting of CO₂R²⁰, —CONR⁷R⁸, and aryl in that aryl is optionally substituted with from 1 to 2 substituents independently selected from the group consisting of halo, C₁₋₃ alkyl, CF₃ or OR¹⁰;
R⁷ is selected from the group consisting of hydrogen, and C₁₋₈ alkyl that is optionally substituted with one substituent selected from the group consisting of halo, CF₃, CN or OR²⁰;
R⁸ is selected from the group consisting of hydrogen and C₁₋₃ alkyl; and R²⁰ is selected from the group consisting of hydrogen and C₁₋₄ alkyl.
   In this more preferred embodiment, R⁸ is preferably hydrogen, R⁷ is preferably selected from the group consisting of hydrogen, and C₁₋₃, and R²⁰ is preferably selected from the group consisting of hydrogen and C₁₋₄ alkyl.

Specific useful compounds are selected from
ethyl 1-{9-[(4S,2R,3R,5R)-3,4-dihydroxy-5-(hydroxymethyl)oxolan-2-yl]-6-aminopurin-2-yl}pyrazole-4-carboxylate,
(4S,2R,3R,5R)-2-{6-amino-2-[4-(4-chlorophenyl)pyrazolyl]purin-9-yl}-5-(hydroxymethyl)oxolane-3,4-diol,
(4S,2R,3R,5R)-2-{6-amino-2-[4-(4-methoxyphenyl)pyrazolyl]purin-9-yl}-5-(hydroxymethyl)oxolane-3,4-diol,
(4S,2R,3R,5R)-2-{6-amino-2-[4-(4-methylphenyl)pyrazolyl]purin-9-yl}-5-(hydroxymethyl)oxolane-3,4-diol,
(1-{9-[(4S,2R,3R,5R)-3,4-dihydroxy-5-(hydroxymethyl)oxolan-2-yl]-6-aminopurin-2-yl}pyrazol-4-yl)-N-methylcarboxamide,
1-{9-[(4S,2R,3R,5R)-3,4-dihydroxy-5-(hydroxymethyl)oxolan-2-yl]-6-aminopurin-2-yl}pyrazole-4-carboxylic acid,
(1-{9-[(4S,2R,3R,5R)-3,4-dihydroxy-5-(hydroxymethyl)oxolan-2-yl]-6-aminopurin-2-yl}pyrazol-4-yl)-N,N-dimethylcarboxamide,
(1-{9-[(4S,2R,3R,5R)-3,4-dihydroxy-5-(hydroxymethyl)oxolan-2-yl]-6-aminopurin-2-yl}pyrazol-4-yl)-N-ethylcarboxamide,
1-{9-[(4S,2R,3R,5R)-3,4-dihydroxy-5-(hydroxymethyl)oxolan-2-yl]-6-aminopurin-2-yl}pyrazole-4-carboxamide,
1-{9-[(4S,2R,3R,5R)-3,4-dihydroxy-5-(hydroxymethyl)oxolan-2-yl]-6-aminopurin-2-yl}pyrazol-4-yl)-N-(cyclopentylmethyl)carboxamide,
(1-{9-[(4S,2R,3R,5R)-3,4-dihydroxy-5-(hydroxymethyl)oxolan-2-yl]-6-aminopurin-2-yl}pyrazol-4-yl)-N-[(4-chlorophenyl)methyl]carboxamide,
ethyl 2-[(1-{9-[(4S,2R,3R,5R)-3,4-dihydroxy-5-(hydroxymethyl)oxolan-2-yl]-6-aminopurin-2-yl}pyrazol-4-yl)carbonylamino]acetate, and mixtures thereof.

A second class of compounds that are potent and selective agonists for the A_{2A} adenosine receptor that are useful in the methods of this invention are 2-adenosine C-pyrazole compounds having the following formula:

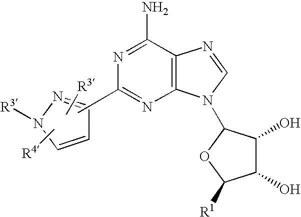

wherein

R¹ is as previously defined;

R^{2′} is selected from the group consisting of hydrogen, C₁₋₁₅ alkyl, C₂₋₁₅ alkenyl, C₂₋₁₅ alkynyl, heterocyclyl, aryl, and heteroaryl, wherein the alkyl, alkenyl, alkynyl, aryl, heterocyclyl, and heteroaryl substituents are optionally substituted with from 1 to 3 substituents independently selected from the group consisting of halo, NO₂, heterocyclyl, aryl, heteroaryl, CF₃, CN, OR²⁰, SR²⁰, N(R²⁰)₂, S(O)R²², SO₂R²², SO₂N(R²⁰)₂, SO₂NR²⁰COR²², SO₂NR²⁰CO₂R²², SO₂NR²⁰CON(R²⁰)₂, N(R²⁰)₂ NR²⁰COR²², NR²⁰CO₂R²², NR²⁰CON(R²⁰)₂, NR²⁰C(NR²⁰)NHR²³, COR²⁰, CO₂R²⁰, CON(R²⁰)₂, CONR²⁰SO₂R²², NR²⁰SO₂R²², SO₂NR²⁰CO₂R²², OCONR²⁰SO₂R²², OC(O)R²⁰, C(O)OCH₂OC(O)R²⁰, and OCON(R²⁰)₂ and wherein each optional heteroaryl, aryl, and heterocyclyl substituent is optionally substituted with halo, NO₂, alkyl, CF₃, amino, mono- or di-alkylamino, alkyl or aryl or heteroaryl amide, NCOR²², NR²⁰SO₂R²², COR²⁰, CO₂R²⁰, CON(R²⁰)₂, NR²⁰CON(R²⁰)₂, OC(O)R²⁰, OC(O)N(R²⁰)₂, SR²⁰, S(O)R²², SO₂R²², SO₂N(R²⁰)₂, CN, or OR²⁰;

R^{3′}, R^{4′} are individually selected from the group consisting of hydrogen, C₁₋₁₅ alkyl, C₂₋₁₅ alkenyl, C₂₋₁₅ alkynyl, heterocyclyl, aryl, and heteroaryl, halo, NO₂, CF₃, CN, OR²⁰, SR²⁰, N(R²⁰)₂, S(O)R²², SO₂R²², SO₂N(R²⁰)₂, SO₂NR²⁰COR²², SO₂NR²⁰CO₂R²², SO₂NR²⁰CON(R²⁰)₂, N(R²⁰)₂ NR²⁰COR²², NR²¹CO₂R²², NR²⁰CON(R²⁰)₂, NR²⁰C(NR²⁰)NHR²³, COR²⁰, CO₂R²⁰, CON(R²⁰)₂, CONR²⁰SO₂SR²², NR²⁰SO₂R²², SO₂NR²⁰CO₂R²², OCONR²⁰SO₂R²², OC(O)R²⁰, C(O)OCH₂OC(O)R²⁰, and OCON(R²⁰)₂ wherein the alkyl, alkenyl, alkynyl, aryl, heterocyclyl, and heteroaryl substituents are optionally substituted with from 1 to 3 substituents individually selected from the group consisting of halo, NO₂, heterocyclyl, aryl, heteroaryl, CF₃, CN, OR²⁰, SR²⁰, N(R²²)₂, S(O)R²², SO₂R²², SO₂N(R²⁰)₂, SO₂NR²⁰COR²², SO₂NR²⁰CO₂R²², SO₂NR²⁰CON(R²⁰)₂, N(R²⁰)₂ NR²⁰COR²², NR²⁰CO₂R²², NR²⁰CON(R²⁰)₂, NR²⁰C(NR²⁰)NHR²³, COR²⁰, CO₂R²⁰, CON(R²⁰)₂, CONR²⁰SO₂R²², NR²⁰SO₂R²², SO₂NR²⁰CO₂R²², OCONR²⁰SO₂R²², OC(O)R²⁰, C(O)OCH₂OC(O)R²⁰, and OCON(R²⁰)₂ and wherein each optional heteroaryl, aryl, and heterocyclyl substituent is optionally substituted with halo, NO₂, alkyl, CF₃, amino, mono- or di-alkylamino, alkyl or aryl or heteroaryl amide, NCOR²², NR²⁰SO₂R²², COR²⁰, CO₂R²⁰, CON(R²⁰)₂, NR²⁰CON(R²⁰)₂, OC(O)R²⁰, OC(O)N(R²⁰)₂, SR²⁰, S(O)R²², SO₂R²², SO₂N(R²⁰)₂, CN, or OR²⁰; and

R⁵R⁶, R²⁰, and R²² are also as previously defined,

with the proviso that when R¹═CH₂OH, R^{3′} is H, R^{4′} is H, the pyrazole ring is attached through C^{4′}, and R^{2′} is not H.

When the compound is selected has one of the following formulas:

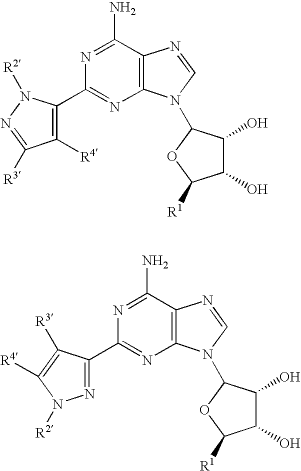

then it is preferred that R¹ is —CH₂OH; R^{2′} is selected from the group consisting of hydrogen, C₁₋₈ alkyl wherein the alkyl is optionally substituted with one substituent independently selected from the group consisting of aryl, CF₃, CN, and wherein each optional aryl substituent is optionally substituted with halo, alkyl, CF₃ or CN; and R^{3′} and R^{4′} are each independently selected from the group consisting of hydrogen, methyl and more preferably, R³ and R⁴ are each hydrogen.

When the compound of this invention has the following formulas:

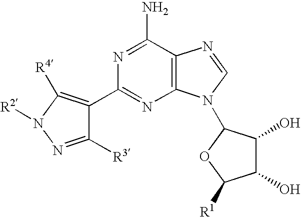

then it is preferred that R¹ is —CH₂OH; R^{2′} is selected from the group consisting of hydrogen, and C₁₋₆ alkyl optionally substituted by phenyl. More preferably, R² is selected from benzyl and pentyl; R³ is selected from the group consisting of hydrogen, C₁₋₆ alkyl, aryl, wherein the alkyl, and aryl substituents are optionally substituted with from 1 to 2 substituents independently selected from the group consisting of halo, aryl, CF₃, CN, and wherein each optional aryl substituent is optionally substituted with halo, alkyl, CF₃ or CN; and R^{4′} is selected from the group consisting of hydrogen and C₁₋₆ alkyl, and more preferably, R^{4′} is selected from hydrogen and methyl.

A more specific class of compounds is selected from the group consisting of
(4S,2R,3R,5R)-2-{6-amino-2-[1-benzylpyrazol-4-yl]purin-9-yl}-5-(hydroxymethyl)oxolane-3,4-diol,
(4S,2R,3R,5R)-2-[6-amino-2-(1-pentylpyrazol-4-yl)purin-9-yl]-5-(hydroxymethyl)oxolane-3,4-diol,
(4S,2R,3R,5R)-2-[6-amino-2-(1-methylpyrazol-4-yl)purin-9-yl]-5-(hydroxymethyl)oxolane-3,4-diol,
(4S,2R,3R,5R)-2-{6-amino-2-[1-(methylethyl)pyrazol-4-yl]purin-9-yl}-5-(hydroxymethyl)oxolane-3,4-diol,
(4S,2R,3R,5R)-2-{6-amino-2-[1-(3-phenylpropyl)pyrazol-4-yl]purin-9-yl}-5-(hydroxymethyl)oxolane-3,4-diol,
(4S,2R,3R,5R)-2-{6-amino-2-[1-(4-t-butylbenzyl)pyrazol-4-yl]purin-9-yl}-5-(hydroxymethyl)oxolane-3,4-diol,
(4S,2R,3R,5R)-2-(6-amino-2-pyrazol-4-ylpurin-9-yl)-5-(hydroxymethyl)oxolane-3,4-diol,
(4S,2R,3R,5R)-2-{6-amino-2-[1-pent-4-enylpyrazol-4-yl]purin-9-yl}-5-(hydroxymethyl)oxolane-3,4-diol,
(4S,2R,3R,5R)-2-{6-amino-2-[1-decylpyrazol-4-yl]purin-9-yl}-5-(hydroxymethyl)oxolane-3,4-diol,
(4S,2R,3R,5R)-2-{6-amino-2-[1-(cyclohexylmethyl)pyrazol-4-yl]purin-9-yl}-5-(hydroxymethyl)oxolane-3,4-diol,
(4S,2R,3R,5R)-2-{6-amino-2-[1-(2-phenylethyl)pyrazol-4-yl]purin-9-yl}-5-(hydroxymethyl)oxolane-3,4-diol,
(4S,2R,3R,5R)-2-{6-amino-2-[1-(3-cyclohexylpropyl)pyrazol-4-yl]purin-9-yl}-5-(hydroxymethyl)oxolane-3,4-diol,
(4S,2R,3R,5R)-2-{6-amino-2-[1-(2-cyclohexylethyl)pyrazol-4-yl]purin-9-yl}-5-(hydroxymethyl)oxolane-3,4-diol, and combinations thereof.

A very useful and potent and selective agonists for the A2A adenosine receptor is Regadenoson or (1-{9-[(4S,2R,3R,5R)-3,4-dihydroxy-5-(hydroxymethyl)oxolan-2-yl]-6-aminopurin-2-yl}pyrazol-4-yl)-N-methylcarboxamide which has the formula:

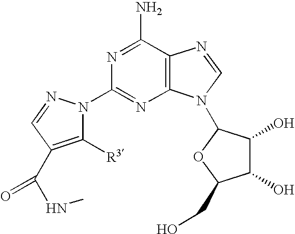

Another preferred compound that is useful as a selective partial A_{2A}-adenosine receptor agonist with a short duration of action is a compound of the formula:

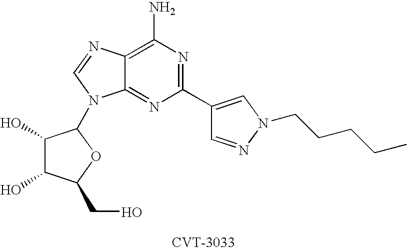

CVT-3033 is particularly useful as an adjuvant in cardiological imaging.

The first and second classes of compounds identified above are described in more detail in U.S. Pat. Nos. 6,403,567 and 6,214,807, the specification of each of which is incorporated herein by reference.

The following definitions apply to terms as used herein.

“Halo” or “Halogen”—alone or in combination means all halogens, that is, chloro (Cl), fluoro (F), bromo (Br), iodo (I).

“Hydroxyl” refers to the group —OH.

“Thiol” or “mercapto” refers to the group —SH.

“Alkyl”—alone or in combination means an alkane-derived radical containing from 1 to 20, preferably 1 to 15, carbon atoms (unless specifically defined). It is a straight chain alkyl, branched alkyl or cycloalkyl. Preferably, straight or branched alkyl groups containing from 1-15, more preferably 1 to 8, even more preferably 1-6, yet more preferably 1-4 and most preferably 1-2, carbon atoms, such as methyl, ethyl, propyl, isopropyl, butyl, t-butyl and the like. The term “lower alkyl” is used herein to describe the straight chain alkyl groups described immediately above. Preferably, cycloalkyl groups are monocyclic, bicyclic or tricyclic ring systems of 3-8, more preferably 3-6, ring members per ring, such as cyclopropyl, cyclopentyl, cyclohexyl, adamantyl and the like. Alkyl also includes a straight chain or branched alkyl group that contains or is interrupted by a cycloalkyl portion. The straight chain or branched alkyl group is attached at any available point to produce a stable compound. Examples of this include, but are not limited to, 4-(isopropyl)-cyclohexylethyl or 2-methyl-cyclopropylpentyl. A substituted alkyl is a straight chain alkyl, branched alkyl, or cycloalkyl group defined previously, independently substituted with 1 to 3 groups or substituents of halo, hydroxy, alkoxy, alkylthio, alkylsulfinyl, alkylsulfonyl, acyloxy, aryloxy, heteroaryloxy, amino optionally mono- or di-substituted with alkyl, aryl or heteroaryl groups, amidino, urea optionally substituted with alkyl, aryl, heteroaryl or heterocyclyl groups, aminosulfonyl optionally N-mono- or N,N-di-substituted with alkyl, aryl or heteroaryl groups, alkylsulfonylamino, arylsulfonylamino, heteroarylsulfonylamino, alkylcarbonylamino, arylcarbonylamino, heteroarylcarbonylamino, or the like.

“Alkenyl”—alone or in combination means a straight, branched, or cyclic hydrocarbon containing 2-20, preferably 2-17, more preferably 2-10, even more preferably 2-8, most preferably 2-4, carbon atoms and at least one, preferably 1-3, more preferably 1-2, most preferably one, carbon to carbon double bond. In the case of a cycloalkyl group, conjugation of more than one carbon to carbon double bond is not such as to confer aromaticity to the ring. Carbon to carbon double bonds may be either contained within a cycloalkyl portion, with the exception of cyclopropyl, or within a straight chain or branched portion. Examples of alkenyl groups include ethenyl, propenyl, isopropenyl, butenyl, cyclohexenyl, cyclohexenylalkyl and the like. A substituted alkenyl is the straight chain alkenyl, branched alkenyl or cycloalkenyl group defined previously, independently substituted with 1 to 3 groups or substituents of halo, hydroxy, alkoxy, alkylthio, alkylsulfinyl, alkylsulfonyl, acyloxy, aryloxy, heteroaryloxy, amino optionally mono- or di-substituted with alkyl, aryl or heteroaryl groups, amidino, urea optionally substituted with alkyl, aryl, heteroaryl or heterocyclyl groups, aminosulfonyl optionally N-mono- or N,N-di-substituted with alkyl, aryl or heteroaryl groups, alkylsulfonylamino, arylsulfonylamino, heteroarylsulfonylamino, alkylcarbonylamino, arylcarbonylamino, heteroarylcarbonylamino, carboxy, alkoxycarbonyl, aryloxycarbonyl, heteroaryloxycarbonyl, or the like attached at any available point to produce a stable compound.

“Alkynyl”—alone or in combination means a straight or branched hydrocarbon containing 2-20, preferably 2-17, more preferably 2-10, even more preferably 2-8, most preferably 2-4, carbon atoms containing at least one, preferably one, carbon to carbon triple bond. Examples of alkynyl groups include ethynyl, propynyl, butynyl and the like. A substituted alkynyl refers to the straight chain alkynyl or branched alkenyl defined previously, independently substituted with 1 to 3 groups or substituents of halo, hydroxy, alkoxy, alkylthio, alkylsulfinyl, alkylsulfonyl, acyloxy, aryloxy, heteroaryloxy, amino optionally mono- or di-substituted with alkyl, aryl or heteroaryl groups, amidino, urea optionally substituted with alkyl, aryl, heteroaryl or heterocyclyl groups, aminosulfonyl optionally N-mono- or N,N-di-substituted with alkyl, aryl or heteroaryl groups, alkylsulfonylamino, arylsulfonylamino, heteroarylsulfonylamino, alkylcarbonylamino, arylcarbonylamino, heteroarylcarbonylamino, or the like attached at any available point to produce a stable compound.

“Alkyl alkenyl” refers to a group —R—CR′═CR′″ R″″, where R is lower alkyl, or substituted lower alkyl, R′, R′″, R″″ may independently be hydrogen, halogen, lower alkyl, substituted lower alkyl, acyl, aryl, substituted aryl, hetaryl, or substituted hetaryl as defined below.

“Alkyl alkynyl” refers to a groups —RC□CR′ where R is lower alkyl or substituted lower alkyl, R′ is hydrogen, lower alkyl, substituted lower alkyl, acyl, aryl, substituted aryl, hetaryl, or substituted hetaryl as defined below.

“Alkoxy” denotes the group —OR, where R is lower alkyl, substituted lower alkyl, acyl, aryl, substituted aryl, aralkyl, substituted aralkyl, heteroalkyl, heteroarylalkyl, cycloalkyl, substituted cycloalkyl, cycloheteroalkyl, or substituted cycloheteroalkyl as defined.

“Alkylthio” denotes the group —SR, —S(O) n=1-2-R, where R is lower alkyl, substituted lower alkyl, aryl, substituted aryl, aralkyl or substituted aralkyl as defined herein.

“Acyl” denotes groups —C(O)R, where R is hydrogen, lower alkyl substituted lower alkyl, aryl, substituted aryl and the like as defined herein.

“Aryloxy” denotes groups —OAr, where Ar is an aryl, substituted aryl, heteroaryl, or substituted heteroaryl group as defined herein.

“Amino” denotes the group NRR′, where R and R′ may independently by hydrogen, lower alkyl, substituted lower alkyl, aryl, substituted aryl, hetaryl, or substituted hetaryl as defined herein or acyl.

“Amido” denotes the group —C(O)NRR′, where R and R′ may independently by hydrogen, lower alkyl, substituted lower alkyl, aryl, substituted aryl, hetaryl, substituted hetaryl as defined herein.

“Carboxyl” denotes the group —C(O)OR, where R is hydrogen, lower alkyl, substituted lower alkyl, aryl, substituted aryl, hetaryl, and substituted hetaryl as defined herein.

“Aryl”—alone or in combination means phenyl or naphthyl optionally carbocyclic fused with a cycloalkyl of preferably 5-7, more preferably 5-6, ring members and/or optionally substituted with 1 to 3 groups or substituents of halo, hydroxy, alkoxy, alkylthio, alkylsulfinyl, alkylsulfonyl, acyloxy, aryloxy, heteroaryloxy, amino optionally mono- or di-substituted with alkyl, aryl or heteroaryl groups, amidino, urea optionally substituted with alkyl, aryl, heteroaryl or heterocyclyl groups, aminosulfonyl optionally N-mono- or N,N-di-substituted with alkyl, aryl or heteroaryl groups, alkylsulfonylamino, arylsulfonylamino, heteroarylsulfonylamino, alkylcarbonylamino, arylcarbonylamino, heteroarylcarbonylamino, or the like.

“Substituted aryl” refers to aryl optionally substituted with one or more functional groups, e.g., halogen, lower alkyl, lower alkoxy, alkylthio, acetylene, amino, amido, carboxyl, hydroxyl, aryl, aryloxy, heterocycle, hetaryl, substituted hetaryl, nitro, cyano, thiol, sulfamido and the like.

“Heterocycle” refers to a saturated, unsaturated, or aromatic carbocyclic group having a single ring (e.g., morpholino, pyridyl or furyl) or multiple condensed rings (e.g., naphthpyridyl, quinoxalyl, quinolinyl, indolizinyl or benzo[b]thienyl) and having at least one hetero atom, such as N, O or S, within the ring, which can optionally be unsubstituted or substituted with, e.g., halogen, lower alkyl, lower alkoxy, alkylthio, acetylene, amino, amido, carboxyl, hydroxyl, aryl, aryloxy, heterocycle, hetaryl, substituted hetaryl, nitro, cyano, thiol, sulfamido and the like.

“Heteroaryl”—alone or in combination means a monocyclic aromatic ring structure containing 5 or 6 ring atoms, or a bicyclic aromatic group having 8 to 10 atoms, containing one or more, preferably 1-4, more preferably 1-3, even more preferably 1-2, heteroatoms independently selected from the group O, S, and N, and optionally substituted with 1 to 3 groups or substituents of halo, hydroxy, alkoxy, alkylthio, alkylsulfinyl, alkylsulfonyl, acyloxy, aryloxy, heteroaryloxy, amino optionally mono- or di-substituted with alkyl, aryl or heteroaryl groups, amidino, urea optionally substituted with alkyl, aryl, heteroaryl or heterocyclyl groups, aminosulfonyl optionally N-mono- or N,N-di-substituted with alkyl, aryl or heteroaryl groups, alkylsulfonylamino, arylsulfonylamino, heteroarylsulfonylamino, alkylcarbonylamino, arylcarbonylamino, heteroarylcarbonylamino, or the like. Heteroaryl is also intended to include oxidized S or N, such as sulfinyl, sulfonyl and N-oxide of a tertiary ring nitrogen. A carbon or nitrogen atom is the point of attachment of the heteroaryl ring structure such that a stable aromatic ring is retained. Examples of heteroaryl groups are pyridinyl, pyridazinyl, pyrazinyl, quinazolinyl, purinyl, indolyl, quinolinyl, pyrimidinyl, pyrrolyl, oxazolyl, thiazolyl, thienyl, isoxazolyl, oxathiadiazolyl, isothiazolyl, tetrazolyl, imidazolyl, triazinyl, furanyl, benzofuryl, indolyl and the like. A substituted heteroaryl contains a substituent attached at an available carbon or nitrogen to produce a stable compound.

“Heterocyclyl”—alone or in combination means a non-aromatic cycloalkyl group having from 5 to 10 atoms in which from 1 to 3 carbon atoms in the ring are replaced by heteroatoms of O, S or N, and are optionally benzo fused or fused heteroaryl of 5-6 ring members and/or are optionally substituted as in the case of cycloalkyl. Heterocycyl is also intended to include oxidized S or N, such as sulfinyl, sulfonyl and N-oxide of a tertiary ring nitrogen. The point of attachment is at a carbon or nitrogen atom. Examples of heterocyclyl groups are tetrahydrofuranyl, dihydropyridinyl, piperidinyl, pyrrolidinyl, piperazinyl, dihydrobenzofuryl, dihydroindolyl, and the like. A substituted heterocyclyl contains a substituent nitrogen attached at an available carbon or nitrogen to produce a stable compound.

“Substituted heteroaryl” refers to a heterocycle optionally mono or poly substituted with one or more functional groups, e.g., halogen, lower alkyl, lower alkoxy, alkylthio, acetylene, amino, amido, carboxyl, hydroxyl, aryl, aryloxy, heterocycle, substituted heterocycle, hetaryl, substituted hetaryl, nitro, cyano, thiol, sulfamido and the like.

“Aralkyl” refers to the group —R—Ar where Ar is an aryl group and R is lower alkyl or substituted lower alkyl group. Aryl groups can optionally be unsubstituted or substituted with, e.g., halogen, lower alkyl, alkoxy, alkylthio, acetylene, amino, amido, carboxyl, hydroxyl, aryl, aryloxy, heterocycle, substituted heterocycle, hetaryl, substituted hetaryl, nitro, cyano, thiol, sulfamido and the like.

“Heteroalkyl” refers to the group —R-Het where Het is a heterocycle group and R is a lower alkyl group. Heteroalkyl groups can optionally be unsubstituted or substituted with e.g., halogen, lower alkyl, lower alkoxy, alkylthio, acetylene, amino, amido, carboxyl, aryl, aryloxy, heterocycle, substituted heterocycle, hetaryl, substituted hetaryl, nitro, cyano, thiol, sulfamido and the like.

“Heteroarylalkyl” refers to the group —R-HetAr where HetAr is an heteroaryl group and R lower alkyl or substituted lower alkyl. Heteroarylalkyl groups can optionally be unsubstituted or substituted with, e.g., halogen, lower alkyl, substituted lower alkyl, alkoxy, alkylthio, acetylene, aryl, aryloxy, heterocycle, substituted heterocycle, hetaryl, substituted hetaryl, nitro, cyano, thiol, sulfamido and the like.

“Cycloalkyl” refers to a divalent cyclic or polycyclic alkyl group containing 3 to 15 carbon atoms.

“Substituted cycloalkyl” refers to a cycloalkyl group comprising one or more substituents with, e.g., halogen, lower alkyl, substituted lower alkyl, alkoxy, alkylthio, acetylene, aryl, aryloxy, heterocycle, substituted heterocycle, hetaryl, substituted hetaryl, nitro, cyano, thiol, sulfamido and the like.

“Cycloheteroalkyl” refers to a cycloalkyl group wherein one or more of the ring carbon atoms is replaced with a heteroatom (e.g., N, O, S or P).

Substituted cycloheteroalkyl” refers to a cycloheteroalkyl group as herein defined which contains one or more substituents, such as halogen, lower alkyl, lower alkoxy, alkylthio, acetylene, amino, amido, carboxyl, hydroxyl, aryl, aryloxy, heterocycle, substituted heterocycle, hetaryl, substituted hetaryl, nitro, cyano, thiol, sulfamido and the like.

“Alkyl cycloalkyl” denotes the group —R-cycloalkyl where cycloalkyl is a cycloalkyl group and R is a lower alkyl or substituted lower alkyl. Cycloalkyl groups can optionally be unsubstituted or substituted with e.g. halogen, lower alkyl, lower alkoxy, alkylthio, acetylene, amino, amido, carboxyl, hydroxyl, aryl, aryloxy, heterocycle, substituted heterocycle, hetaryl, substituted hetaryl, nitro, cyano, thiol, sulfamido and the like.

“Alkyl cycloheteroalkyl” denotes the group —R-cycloheteroalkyl where R is a lower alkyl or substituted lower alkyl. Cycloheteroalkyl groups can optionally be unsubstituted or substituted with e.g. halogen, lower alkyl, lower alkoxy, alkylthio, amino, amido, carboxyl, acetylene, hydroxyl, aryl, aryloxy, heterocycle, substituted heterocycle, hetaryl, substituted hetaryl, nitro, cyano, thiol, sulfamido and the like.

The terms “sub-maximal exercise” and “low level exercise” are used to refer to any exercise regimen designed to be one that could be performed by most patients who would be referred for pharmacologic testing (i.e., those who would not be expected to achieve 85% or more of maximum predicted heart rate with exercise) but one that would still elicit the desired sympathetic response.

The following Example is representative of the invention, but is not to be construed as limiting the scope of the claims.

### EXAMPLES

### Example 1

### Methods

### Study Design

In this multicenter study, subjects requiring pharmacologic MPI based on clinical criteria received adenosine infusion (Astellas Pharma, Inc.), 140 mcg/kg/min over 6 min in the supine position (AdenoSup), following enrollment and were then randomized (2:1) in a double-blind manner to a novel protocol (RegEx) consisting of 4 min of sub-maximal exercise (1.7 mph at 0% grade) with bolus intravenous injection of 400 mcg Regadenoson at 1.5 min and ^{99m}Technetium-Sestamibi at 2 min or matching placebos (PlcEx).

Prior to randomization, patients were stratified based on the presence of reversible perfusion defects, defined as a two or more segments with a stress score >rest score and a stress score >2 on a 5-category scale, as interpreted by a board-certified nuclear cardiologist at each site. The 5-category scale, used both for stratifying patients and for evaluation of perfusion defects on study, was as follows: 0=normal; 1=mild reduction in tracer uptake, not definitely abnormal; 2=moderate reduction in uptake, definitely abnormal; 3=severe reduction in uptake; 4=absent uptake.

The primary objective was to assess the overall safety of Regadenoson in patients undergoing low-level stress by comparing hemodynamic, cardiac rhythm and adverse effects of the 3 protocols. In addition, patient acceptance was determined by comparing patient comfort and test protocol preference using questionnaires. Three blinded expert readers independently interpreted randomly presented perfusion scans at a nuclear core lab (Services NucMed, Montreal, Canada). Image quality was compared between AdenoSup and RegEx by computation of heart-to-liver and heart-to-gut ratios and the readers' visual assessment of overall image quality and image quality with respect to subdiaphragmatic interference specifically. A 17-segment MPI model was used by the core lab readers, to compare the extent of the perfusion defect between RegEx and AdenoSup quantitatively and also qualitatively with side-by-side visual comparison. Patients were required to abstain from methylxanthine-containing foods and beverages for 12 hours prior to receiving study drug and adenosine. The protocol was approved by an institutional review board and all patients provided written informed consent.

### Imaging Protocols

Nuclear imaging was performed using either a dual isotope protocol or a two-day ^{99m}Technetium-Sestainibi protocol at the investigators' discretion. However, men with body weight >220 pounds and body mass index >30 kg/m2 and women with body weight >200 pounds and body mass index >30 kg/m2 were to undergo the two day protocol. The single photon emission computed tomography (SPECT) imaging was standardized for image acquisition and transmittal in accordance with the American Society of Nuclear Cardiology guidelines. The protocol required an extra ˜8 mSv radiation to the patients in the study arm, and none to the patients in the control (placebo exercise) arm.

The dual isotope protocol was performed over 2 separate days. On the first day, patients were to have a rest scan with ²⁰¹Thallium followed by a ^{99m}Technetium-Sestamibi adenosine-supine MPI; on a subsequent day, patients underwent a ^{99m}Technetium-Sestamibi study drug (i.e., regadenoson or placebo) sub-maximal treadmill exercise MPI.

The multi-day ^{99m}Technetium-Sestamibi was performed over 3 days. On the first day, patients were to have a ^{99m}Technetium-Sestamibi adenosine supine MPI or ^{99m}Technetium-Sestamibi rest scan. On the second day, the patient was to have either the rest or stress, whichever was not received on the first day and, on the third day, the patient had a ^{99m}Technetium-Sestamibi study drug (i.e., Regadenoson or placebo) sub-maximal treadmill exercise MPI. The second and third days were not necessarily consecutive to the first day.

The stress MPI scans were to be performed 60±10 minutes after the start of adenosine or study drug. Regions of interest, defined as the entire left ventricle, a 25 square pixel area over the right upper lobe of the liver excluding the common bile duct, and a 5×5-pixel square area of the gut beginning 5 pixels inferior to the mid-inferior wall of the heart were identified from a 60-second planar view of the thorax and abdomen, prior to each SPECT imaging. A region of interest in the gut area below the heart was chosen because of the potential deleterious effect on interpretation of inferior wall perfusion. Specifically, either direct overlap of the gut or activity immediately below the inferior wall greater than the inferior wall itself can result in an artifactual subtraction of counts from the inferior wall intrinsic to commonly used edge-detection software.

### Patients

To be enrolled in the study, patients must have been >18 years of age, required a clinically-indicated adenosine pharmacologic stress SPECT MPI, and were judged capable of exercising sufficiently to perform the study drug low level exercise. Female patients who were pregnant, breastfeeding, or of childbearing potential were not included. The primary exclusion criteria were as follows:
1) History of coronary revascularization by either percutaneous coronary intervention or coronary artery bypass graft or documented history of acute myocardial infarction or unstable angina within 3 months;
2) Change within 7 days of adenosine-supine MPI of medications that may affect the rate-pressure product or anticipated changes in such medications during the study;
3) Uncontrolled hypertension (i.e., >200/120 mm Hg);
4) Known hypertrophic cardiomyopathy with obstruction or severe aortic stenosis;
5) Decompensated congestive heart failure or cardiac transplantation;
6) A history of sick sinus syndrome or greater than 1st degree AV block, except in patients who had a functional artificial pacemaker or in whom these conditions occurred due to a temporary condition that now no longer exists;
7) Asthma or other bronchospastic reactive airway disease; and
8) Current use of dipyridamole, aminophylline use within 24 hours, or theophylline use within 48 hours.

### Statistical Methods

Changes in blood pressure, heart rate, and ECG intervals were computed over time and compared (Regadenoson vs. placebo) using repeated measures, mixed-model ANOVA. The incidence of symptomatic hypotension, systolic blood pressure decreases of >20 mm Hg, ECG abnormalities, and severe or related adverse events was compared using Fisher's exact test. The quality of nuclear MPI scans following regadenoson and low-level exercise was compared to adenosine-supine MPI scans of the same subject using the sign test. Radiotracer target-to-background ratios were computed and compared between the two imaging regimens using the Wilcoxon signed ranks test. Semi-quantitative scoring of perfusion defects (Summed Stress Score (SSS), Summed Difference Score (SDS), etc.) was conducted using a 17-segment polar map and the quality of agreement between the two imaging regimens was assessed using

Cohen's kappa for the categories 0-3, 4-7, 8-11, and >12 (SSS) and 0-6, 7-13, and >14 (SDS). The number of segments with reversible perfusion defects was defined as the median number across the three readers. Subject comfort and tolerability were assessed using a 4-point scale and the regimens compared using a Cochran-Mantel-Haenszel test of equality of mean scores. Data are expressed as mean (SD) unless otherwise specified. Statistical analyses were conducted using SAS version 9.1. Statistical significance was defined as a p-value of <0.05.

### Results

A total of 62 patients were enrolled in the study and underwent adenosine MPI; 60 of these patients were subsequently randomized to either Regadenoson MPI (n=39) or placebo MPI (n=21). Two patients were not randomized following adenosine MPI and were prematurely terminated from the study because of the initiation of a β-blocker within 6 days prior to the adenosine MPI and elective withdrawal, respectively. Of the 39 patients randomized to Regadenoson MPI, 20 were in the reversible perfusion defects stratum and 19 were in the no reversible perfusion defects stratum; and of the 21 patients randomized to placebo MPI, 10 were in the reversible perfusion defects stratum and 11 were in the no reversible perfusion defects stratum. All 60 randomized patients completed the 6-minute adenosine treatment, were treated with study drug, completed the sub-maximal exercise per protocol, and completed the study.

Patient demographics are shown in Table 1. There was a higher percentage of women among those receiving Regadenoson, compared to those receiving placebo (52% vs. 21%, p=0.011).

TABLE 1
Baseline Characteristics

| | Regadenoson | | Placebo | | | All | |
|---|---|---|---|---|---|---|---|
| Variable | (n = 39) | | (n = 21) | | p-value | (n = 60) | |
| Age (years) | | | | | | | |
| mean (SD) | 70 | (10.3) | 69 | (7.4) | 0.55 | 70 | (9.4) |
| ≧65 | 28 | (72%) | 17 | (81%) | | 45 | (75%) |
| ≧75 | 16 | (41%) | 5 | (24%) | | 21 | (35%) |
| Gender | | | | | | | |
| % Male | 31 | (79%) | 10 | (48%) | 0.011 | 41 | (68%) |
| Race | | | | | | | |
| % Caucasian | 36 | (92%) | 18 | (86%) | 0.42 | 54 | (90%) |
| % Black | 1 | (3%) | 2 | (10%) | | 3 | (5%) |
| % Other | 2 | (6%) | 1 | (5%) | | 3 | (5%) |
| Weight (kg) | | | | | | | |
| Mean (SD) | 86 | (18) | 83 | (13) | 0.67 | 85 | (16) |
| Body Mass Index | | | | | | | |
| (kg/m²) | | | | | | | |
| Mean (SD) | 29 | (5) | 29 | (3) | 0.47 | 29 | (5) |
| Range | 21-42 | | 23-37 | | | 21-42 | |
| History of | 31 | (79%) | 18 | (86%) | 0.55 | 49 | (82%) |
| Coronary Artery | | | | | | | |
| Disease | | | | | | | |
| History of | 15 | (38%) | 4 | (19%) | 0.12 | 19 | (32%) |
| Diabetes Mellitus | | | | | | | |
| History of | 14 | (36%) | 4 | (19%) | 0.17 | 18 | (30%) |
| Congestive Heart | | | | | | | |
| Failure | | | | | | | |
| History of | 33 | (85%) | 19 | (90%) | 0.52 | 52 | (87%) |
| Hypertension | | | | | | | |

Chi-squared test p-values are shown for categorical variables and Wilcoxon's rank sum test p-values for continuous variables. For race, the proportion of Caucasian patients is compared. “History of Congestive Heart Failure” includes patients with medical histories of congestive heart failure, left ventricular dysfunction, cardiomyopathy, and cardiomegaly.

The frequency of use of cardiovascular drugs (Table 2) in the study population is consistent with the high frequency of pre-existing comorbidities including coronary artery disease, hypertension, congestive heart failure, and diabetes (Table 1).

TABLE 2
Selected Baseline Medications

| | | | | | All | |
|---|---|---|---|---|---|---|
| | Regadenoson | | Placebo | | Subjects | |
| | (n = 39) | | (n = 21) | | (n = 60) | |
| Lipid Lowering Drugs | | | | | | |
| HMG-CoA Reductase | 32 | (82%) | 18 | (86%) | 52 | (84%) |
| Inhibitors | | | | | | |
| Other Drugs for | 18 | (46%) | 11 | (52%) | 29 | (48%) |
| Hyperlipidemia | | | | | | |
| Renin-Angiotensin- | | | | | | |
| Aldosterone System | | | | | | |
| Inhibitors | | | | | | |
| Angiotensin II Receptor | 5 | (13%) | 9 | (43%) | 14 | (23%) |
| Blockers (ARB) and ARB/ | | | | | | |
| Diuretic Combination Drugs | | | | | | |
| Angiotensin Converting | 25 | (64%) | 6 | (29%) | 33 | (53%) |
| Enzyme Inhibitors | | | | | | |
| Diuretics | 12 | (31%) | 4 | (19%) | 27 | (45%) |
| Dihydropyridine Calcium | 7 | (18%) | 5 | (24%) | 12 | (19%) |
| Channel Blockers | | | | | | |
| Diltiazem | 1 | (3%) | 1 | (5%) | 2 | (3%) |
| Adrenergic Receptor | | | | | | |
| Antagonists | | | | | | |
| β-Blockers | 22 | (56%) | 16 | (76%) | 38 | (47%) |
| α and β-blocking | 4 | (10%) | 3 | (14%) | 7 | (12%) |
| Agents (carvedilol) | | | | | | |
| α Adrenoceptor | 8 | (21%) | 2 | (10%) | 10 | (16%) |
| Antagonists | | | | | | |
| Platelet Aggregation | 29 | (74%) | 16 | (76%) | 45 | (75%) |
| Inhibitors | | | | | | |
| Nitrates | 7 | (18%) | 4 | (19%) | 11 | (18%) |
| Digoxin | 3 | (8%) | 2 | (10%) | 5 | (8%) |
| Anti-Arrhythmics | | | | | | |
| Class IC | 1 | (3%) | 1 | (5%) | 2 | (3%) |
| Class III (amiodarone) | 0 | | 1 | (5%) | 1 | (2%) |
| Warfarin | 2 | (11%) | 2 | (18%) | 4 | (13%) |
| Anti-Diabetic Drugs | | | | | | |
| Insulin | 5 | (13%) | 1 | (3%) | 6 | (10%) |
| Non-Insulin Drugs | 11 | (28%) | 4 | (19%) | 15 | (25%) |

For categories containing multiple drugs, counts shown represent the number of unique patients receiving a given category of drugs

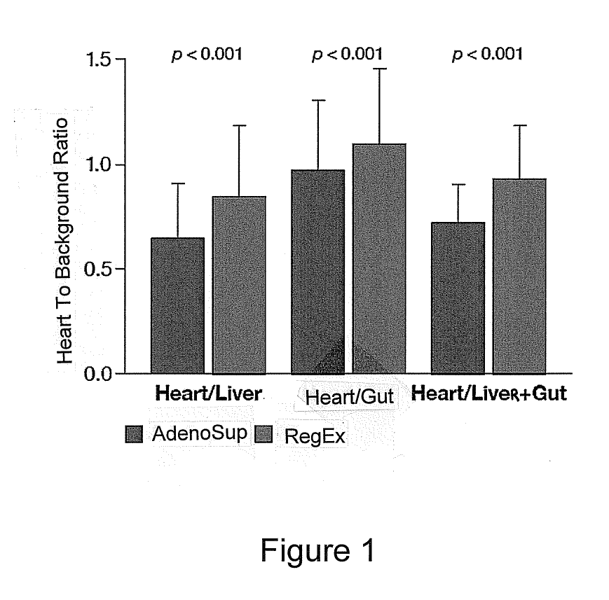

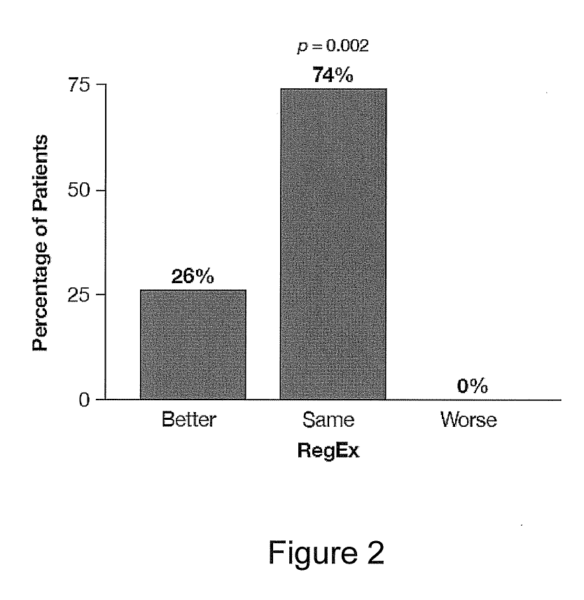

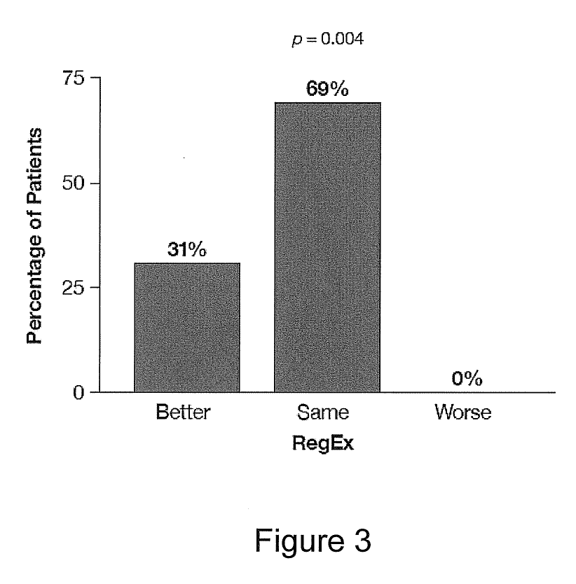

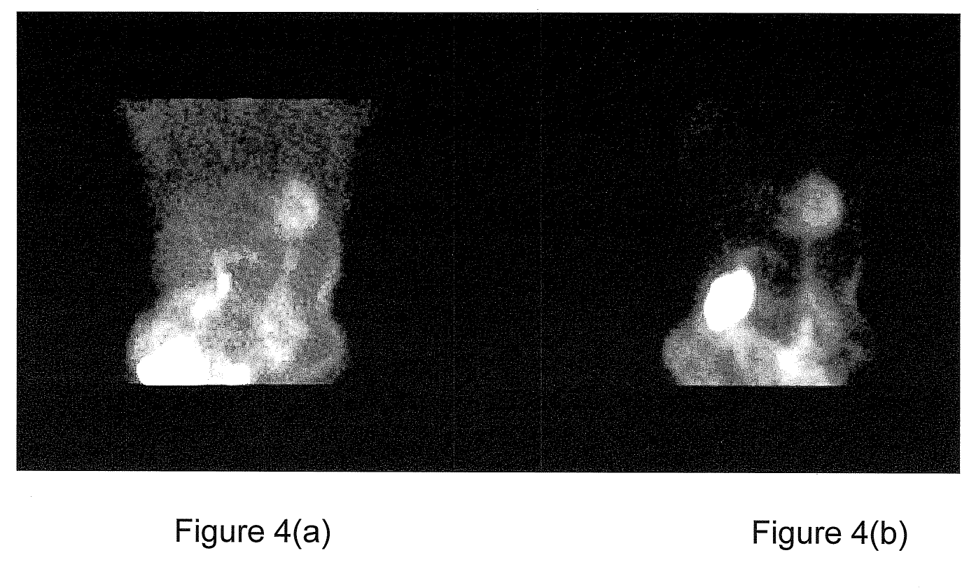

Target (heart)-to-background ratios (heart-to-liver, heart-to-gut, and heart-to-liver+gut) were significantly higher on the RegEx scans compared to the AdenoSup scans (FIG. 1). The mean (SD) heart-to-liver ratio of RegEx and AdenoSup amongst the 39 patients undergoing both of these scans was 0.85 (0.34) and 0.65 (0.26), respectively, p<0.001. The comparable values for the mean heart-to-gut ratio were 1.1 (0.36) vs. 0.97 (0.34), p<0.001, respectively, and those for the heart-to-liver+gut ratio were 0.93 (0.26) and 0.72 (0.18), respectively, p<0.001. In side-by-side comparisons of studies from the 39 patients who received AdenoSup and were subsequently randomized to RegEx, the latter had significantly better overall image quality (p=0.002) and image quality with respect to subdiaphragmatic interference (p=0.004) (FIGS. 2, 3, and 4). A representative example of the difference in image quality and target-to background ratios is shown in FIG. 4. Reversible perfusion defects were detected in 25 out of 39 (64.1%) patients on RegEx and 20 out of 39 (51.3%) of the same patients on AdenoSup [kappa=0.64, 95% CI, 0.40, 0.87].

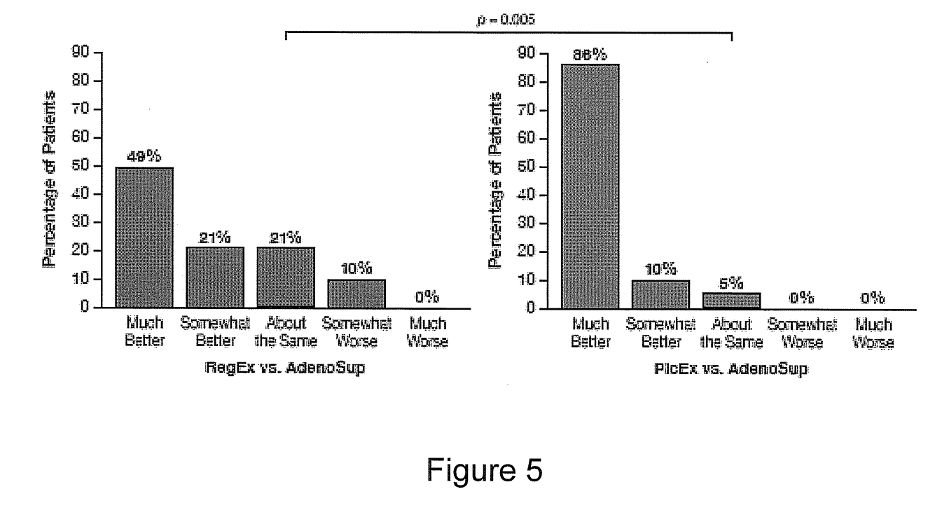

Both RegEx and PlcEx were well tolerated: 59% and 95% of patients, respectively, reported the tests as being “comfortable” and 41% and 5%, respectively, as being “a little uncomfortable” on a 4-point scale. No patients reported being very uncomfortable or extremely uncomfortable. Compared to those receiving AdenoSup, 70% of patients receiving RegEx and 96% of patients receiving PlcEx felt that the test with exercise was “much better” or “somewhat better” (FIG. 5).

Following AdenoSup, 95% of the 62 patients dosed experienced at least one adverse event, defined as any abnormal sign or symptom, regardless of perceived causality. The corresponding percentages following RegEx (n=39) and PlcEx (n=21) were 77% and 33%, respectively (Table 3). Dyspnea was the only adverse event that occurred with a higher frequency (>10% difference) during RegEx (54%) compared to AdenoSup (41%) (exact McNemar p=0.23).

TABLE 3
Adverse Events Occurring in ≧10% of Patients in Any Group

| | AdenoSup | | | | | | | |
|---|---|---|---|---|---|---|---|---|
| | Later | | Later | | | | | |
| | randomized | | randomized | | | | | |
| | to RegEx | | to PlcEx | | RegEx | | PlcEx | |
| Event | (n = 39) | | (n = 21) | | (n = 39) | | (n = 21) | |
| All | 37 | (95%) | 20 | (95%) | 30 | (77%) | 7 | (33%) |
| All Cardiac | 10 | (26%) | 4 | (19%) | 2 | (5%) | 2 | (10%) |
| Dyspnea | 16 | (41%) | 11 | (52%) | 21 | (54%) | 4 | (19%) |
| Throat | 4 | (10%) | 2 | (10%) | 0 | | 1 | (5%) |
| tightness | | | | | | | | |
| Headache | 12 | (31%) | 9 | (43%) | 9 | (23%) | 1 | (5%) |
| Dizziness | 0 | | 5 | (13%) | 4 | (19%) | 5 | (13%) |
| Paraesthesia | 8 | (21%) | 1 | (5%) | 2 | (5%) | 0 | |
| Pain in Jaw | 0 | | 3 | (14%) | 0 | | 0 | |
| Abdominal | 4 | (10%) | 3 | (14%) | 2 | (5%) | 0 | |
| Pain | | | | | | | | |
| Nausea | 2 | (5%) | 3 | (14%) | 2 | (5%) | 0 | |
| Stomach | 6 | (15%) | 0 | | 1 | (3%) | 0 | |
| Discomfort | | | | | | | | |
| ST-Segment | 6 | (15%) | 4 | (19%) | 6 | (15%) | 1 | (5%) |
| Depression | | | | | | | | |
| Flushing | 19 | (49%) | 11 | (52%) | 5 | (13%) | 0 | |
| Chest | 11 | (28%) | 8 | (38%) | 4 | (10%) | 1 | (5%) |
| Discomfort | | | | | | | | |

| | | | | | | |
|---|---|---|---|---|---|---|
| Chest Pain | 4 | (10%) | 4 | (19%) | 0 | 0 |

AdenoSup, adenosine supine myocardial perfusion imaging (MPI); PlcEX, placebo with exercise MPI; RegEx, regadenoson with exercise MPI.

One patient developed protocol-defined symptomatic hypotension (defined as the development of a sufficient decline in blood pressure that was likely related to simultaneously occurring symptoms that may accompany hypotension) and this occurred following adenosine treatment. Severe adverse events occurred in 4/60 (6.7%) patients following AdenoSup (abdominal pain, chest pain, ST-segment depression, neck pain, headache, and paraesthesia) and in no patient following RegEx or PIcEx. No patient was withdrawn from the study due to an adverse event, and no patient had a serious adverse event.

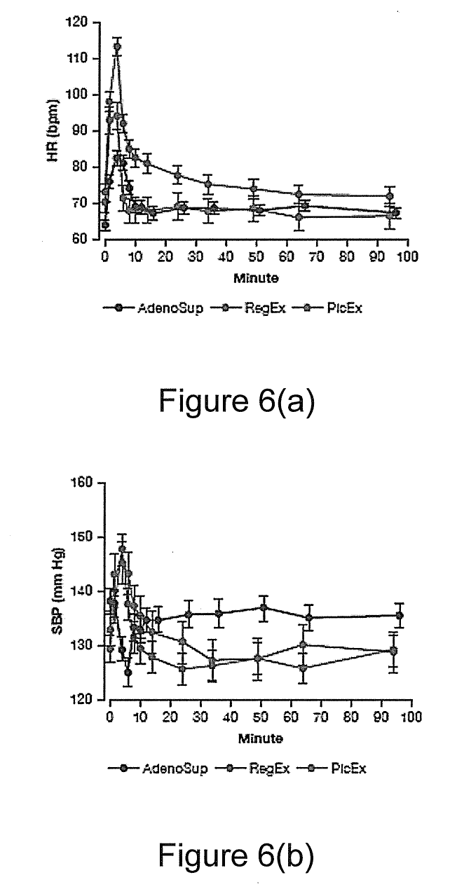

Compared to the peak HR following PIcEx (+28.9 (SE 3.7) bpm) and AdenoSup (+21.0 (SE 2.5) bpm), peak heart rate following RegEx was greater by 13 bpm and 21 bpm, respectively (p=0.006 and <0.001, respectively). This represented a 41.9 (SE 2.7) bpm increase from the resting baseline. The heart rate remained significantly higher during RegEx vs. PIcEx through 24 minutes following start of exercise (FIG. 6A), although by 24 minutes, the HR in the RegEx and PIcEx patients had diminished to +4.6 (SE 1.5) and −1.33 (SE 2.1) bpm, respectively, over the pre-exercise baseline.

During exercise, there were similar and transient mean increases in systolic blood pressure in the RegEx and PlcEx groups (FIG. 6B). Pre-specified analyses of blood pressure, which included change from baseline in mean SBP, change from baseline to nadir SBP, and percentage of patients with a decline in SBP by >20 mm Hg, showed no important differences between RegEx and PlcEx or between RegEx and AdenoSup.

Arrhythmias reported as adverse events or ECG findings occurred in 3 patients following AdenoSup only (atrial fibrillation, atrial tachycardia, and supraventricular arrhythmia) and in 1 patient following RegEx only (supraventricular tachycardia). In 2 patients, arrhythmias occurred following both AdenoSup and RegEx: ventricular tachycardia, ventricular extrasystoles, and “premature ventricular contraction-mediated tachycardia” following adenosine, and ventricular couplet and pacemaker-mediated tachycardia with RegEx.

The effects of AdenoSup, RegEx, and PlcEx on ECG intervals were similar. No occurrences of 2nd degree or higher AV block were observed following RegEx or PlcEx; one patient developed 2nd-degree AV block following AdenoSup.

### Discussion

In this randomized, double-blind, placebo- and active-controlled study including a large proportion of elderly patients with pre-existing coronary artery disease, administration of a Regadenoson bolus of 400 mcg during low-level stress testing was both feasible and well tolerated. Compared to AdenoSup, image quality overall, heart-to-gut and heart-to-liver ratios, and side-by-side comparisons of image quality with respect to subdiaphragmatic interference were significantly better with RegEx.

In addition, sensitivity appeared to be at least as good with the combined low-level exercise—regadenoson protocol compared to the standard resting supine adenosine approach. For example, reversible perfusion defects were detected in 25 of the 39 (64.1%) of patients on RegEx and 20 of the same 39 (51.3%) of patients on AdenoSup (kappa=0.64, 95% CI, 0.40, 0.87). Patients also appeared to tolerate RegEx better than AdenoSup, based on their questionnaire self-reports and the lower frequency and diminished severity of adverse events.

The goals of combining low-level exercise testing with an adenosine agonist pharmacologic MPI agent are three-fold:
1) To increase the tolerability of the pharmacologic agent by inducing a sympathetic response with exercise that offsets the hypotensive and other adverse effects of the adenosine agonists;
2) To obtain the benefits of exercise on enhancing image quality due to a greater relative distribution of blood flow to the heart over the gut and liver;
3) To improve test sensitivity for detecting ischemia.
   Prior to this pilot trial, regadenoson had not been administered in conjunction with exercise. The objective of this study, therefore, was to explore the feasibility, tolerability, and safety of Regadenoson with sub-maximal exercise testing and its effects on image quality, extent of detectable ischemia, and patient tolerability. The exercise regimen created for this trial was designed to be one that could be performed by most patients who would be referred for pharmacologic testing (i.e., those who would not be expected to achieve 85% or more of maximum predicted heart rate with exercise) but one that would still elicit the desired sympathetic response. The testing was performed at a modest speed (1.7 mph) and at 0% grade over 4 minutes. Indeed, all the subjects were able to complete the exercise protocol.

The hemodynamic effects of exercise testing were as expected: there was a transient modest (non-statistically significant) mean increase in systolic blood pressure and a significant increase in mean heart rate relative to supine pharmacologic-only testing with adenosine. The combination of Regadenoson with sub-maximal exercise testing increased the mean maximum heart rate by 16.5 beats per minute over sub-maximal 1 exercise testing with placebo (+40.2 (1.5) bpm on Regadenoson vs. +23.7 (2.1) bpm on placebo). The heart rate difference vs. placebo declined over time such that HR following Regadenoson was <5 bpm higher than the pre-exercise baseline by 24 minutes following the study drug bolus.

In conclusion, this randomized, controlled pilot trial demonstrated for the first time the feasibility and tolerability of administering Regadenoson with low-level exercise. The addition of low-level exercise to Regadenoson appears to provide benefits on image quality, patient acceptance, and side-effects similar to those previously reported for imaging protocols in which exercise is added to adenosine.

What is claimed is:

1. A method of diagnosing myocardial dysfunction during vasodilator induced myocardial stress perfusion imaging in a human patient, comprising administering at least 10 μg of at least one partial A_{2A} adenosine receptor agonist to the patient while the patient is undergoing sub-maximal exercise.

2. The method of claim 1, wherein no more than about 1000 μg of the partial A_{2A} adenosine receptor agonist is administered to the patient.

3. The method of claim 1, wherein the amount of the partial A_{2A} adenosine receptor agonist administered is greater than about 600 μg.

4. The method of claim 1, wherein the amount of the partial A_{2A} adenosine receptor agonist administered is greater than about 100 μg.

5. The method of claim 1, wherein the amount of the partial A_{2A} adenosine receptor agonist administered ranges from about 10 to about 600 μg.

6. The method of claim 5, wherein the A_{2A} adenosine receptor is administered in a single dose.

7. The method of claim 6, wherein the partial A_{2A} adenosine receptor agonist is administered by iv bolus.

8. The method of claim 6, the partial A_{2A} adenosine receptor agonist is administered in less than about 10 seconds.

9. The method of claim 6, wherein the amount of the partial A_{2A} adenosine receptor agonist administered is greater than about 500 μg.

10. The method of claim 6, wherein the partial A_{2A} adenosine receptor agonist is administered in an amount ranging from about 100 μg to about 500 μg.

11. The method of claim 1, wherein the partial A_{2A} adenosine receptor agonist is selected from the group consisting of CVT-3033, Regadenoson, and combinations thereof.

12. A method of diagnosing myocardial dysfunction during vasodilator induced myocardial stress perfusion imaging in a human patient, comprising administering a radionuclide and a partial A_{2A} receptor agonist in an amount ranging from about 10 to about 600 μg while the patient is undergoing sub-maximal exercise, wherein the myocardium is examined for areas of insufficient blood flow following administration of the radionuclide and the partial A_{2A} receptor agonist.

13. The method of claim 12, wherein the myocardium examination begins within about 1 minute from the time the partial A_{2A} adenosine receptor agonist is administered.

14. The method of claim 12, wherein the administration of the partial A_{2A} adenosine receptor agonist causes at least a 2.5 fold increase in coronary blood flow.

15. The method of claim 14, wherein the at least a 2.5 fold increase in coronary blood flow that is achieved within about 1 minute from the administration of the partial A_{2A} adenosine receptor agonist.

16. The method of claim 12, wherein the radionuclide and the partial A_{2A} adenosine receptor agonist are administered separately.

17. The method of claim 12, wherein the radionuclide and the partial A_{2A} adenosine receptor agonist are administered simultaneously.

18. The method of claim 14, wherein the at least a 2.5 fold increase in coronary blood flow is less than about 5 minutes in duration.

19. The method of claim 18, wherein the at least a 2.5 fold increase in coronary blood flow is less than about 3 minutes in duration.

20. A method of diagnosing myocardial dysfunction during vasodilator induced myocardial stress perfusion imaging in a human patient, comprising administering Regadenoson in an amount ranging from about 10 to about 600 μg in a single iv bolus while the patient is undergoing sub-maximal exercise.

21. A method of diagnosing myocardial dysfunction during vasodilator induced myocardial stress perfusion imaging in a human patient, comprising administering Regadenoson in an amount ranging from about 100 to about 500 μg in a single iv bolus while the patient is undergoing sub-maximal exercise.
